(19) United States
(12) Patent Application Publication
(10) Pub. No.: US 2021/0038436 A1
(43) Pub. Date: Feb. 11, 2021

(54) TRANSFER DRESSING FOR EPIDERMAL TRANSFER AND METHOD FOR MAKING SAME

(71) Applicant: KCI Licensing, Inc., San Antonio, TX (US)

(72) Inventors: Kristine M. KIESWETTER, San Antonio, TX (US); Christopher LOCKE, Bournemouth (GB); Prathamesh M. KHARKAR, San Antonio, TX (US); Sandra Osborne GARCIA, San Antonio, TX (US)

(21) Appl. No.: 16/964,960

(22) PCT Filed: Jan. 25, 2019
(86) PCT No.: PCT/US2019/015230

§ 371 Date: Jul. 24, 2020

Related U.S. Application Data
(60) Provisional application No. 62/622,782, filed on Jan. 26, 2018.

(51) Int. Cl. A61F 13/02; A61B 17/322

(52) U.S. Cl. CPC A61F 13/02; A61F 2013/00425; A61B 17/322

(57) ABSTRACT

A transfer dressing that can secure and/or prevent undesired movement of epidermal skin grafts transfer substrates is described. The transfer dressing can include a transfer substrate and one or more adhesive appendages that are configured to secure the transfer substrate to a recipient site (e.g., periwound area) in order to hold the transfer substrate in place and/or facilitate handling of the transfer substrate. The transfer substrate can be comprised of a fenestrated surface configured to facilitate harvesting and transfer of epidermal skin grafts.

### TECHNICAL FIELD

The present disclosure relates generally to an epidermal transfer dressing and, more particularly to apparatus, systems, and methods for securing an epidermal transfer dressing to a recipient site.

### BACKGROUND

Skin is among the largest organs of the human body, representing approximately 16% of an average person's total body weight. Skin can have an important function in body defense because it interfaces with the environment and acts as an anatomical barrier from pathogens and other environmental substances. Skin can also provide a semi-permeable barrier that prevents excessive fluid loss while ensuring that essential nutrients are not washed out of the body. Other functions of skin can include insulation, temperature regulation, and sensation.

Skin tissue can be subject to many forms of damage, such as burns, trauma, disease, and depigmentation. Skin grafts are often used to repair such skin damage. Generally, skin grafting can involve a surgical procedure in which a section of skin is removed from one area of a person's body (autograft from a donor site on a patient's body), removed from another human source (allograft), or removed from another animal (xenograft), and transplanted to a recipient site (e.g., wound site on the patient's body).

However, as with any surgical procedure, skin grafting can involve various risks and complications. Such complications can include graft failure, rejection of the skin graft, bleeding, fluid accumulation, and/or infection at either the donor or recipient site. Additionally, when an autograft is taken from one area of a person's body to produce the graft, some degree of trauma can occur at the donor site. Therefore, if the recipient site is a large wound or otherwise damaged skin region, the trauma at the donor site can be significant.

Various techniques have been developed for harvesting a large number of smaller grafts (e.g., commonly referred to as “micrografts”) to reduce the trauma at the donor site. By removing only a fraction of the skin at a donor site and leaving regions of healthy skin surrounding the excised regions, a large amount of skin for transplantation can be obtained with less discomfort. Micrograft harvesting can also reduce the healing time and risk of infection.

Harvesting of skin grafts can be accomplished in many different ways. One common technique for harvesting a skin graft can involve the application of suction to separate a surface portion of the skin (e.g., the epidermis and a basal cell layer) from the underlying dermis. Harvesting of suction blisters typically also involves a heat source to facilitate blister formation.

Various devices are available for generating and harvesting micrografts. For example, the CelluTome® skin harvester is available from Acelity, Inc. of San Antonio, Tex. The CelluTome® system includes a head that provides a source of reduced pressure (vacuum), and optionally a heater element, and a harvester configured for placement on a target region of a patient's skin. The harvester can form a sealing engagement with the head such that the target region of skin is embraced within an evacuated chamber. The CelluTome® harvester can also include at least one alignment plate having a plurality of holes through which skin blisters can be raised in the presence of negative pressure, and a cutting plate having at least one cutting surface for cleaving skin blisters after they are formed within the chamber.

Typically, micrograft harvesters rely upon a support or transfer substrate or dressing to lift the excised blisters from the device. The transfer substrate can be applied to a recipient site such that the plurality of micrografts are assimilated as transplanted tissue. Transfer substrates can often include an adhesive or tacky surface that is configured to facilitate a) collection of the micrografts from the donor site, and b) application and attachment of the transfer dressing to the recipient site. Generally, transfer substrates having adhesive or tacky coatings on one or both sides can be used. Transfer substrates having double sided tackiness can facilitate the adhesion of the transfer substrate to secondary substrate and/or dressing that may be attached to the tissue surrounding the recipient site (e.g., periwound tissue surrounding a wound) and/or assist in keeping the transfer substrate in place at the recipient site (e.g., keeping the transfer substrate in place at the wound bed).

However, double sided transfer substrates can make handling and placement of the transfer substrate cumbersome since one side of the transfer substrate can adhere to a clinician's hands or gloves during placement. Single sided transfer substrates, which do not offer the benefit of adhering to secondary dressings at the wound site as double sided transfer substrates do, can also bunch-up and self-adhere and/or shuffle to one side of the wound upon being placed within the wound bed. However, since unwanted movements of the transfer substrate can inhibit expansion of the grafts and/or the healing process, it is important for the transfer substrate to remain outstretched to allow for the epidermal skin grafts (ESG) to remain in direct contact with the whole wound at the recipient site.

### SUMMARY

The present disclosure relates to a transfer dressing configured to be secured to a recipient site to prevent undesired movement of epidermal skin grafts transfer substrates. The transfer dressing can include one or more adhesive extensions or appendages that are configured to secure the transfer dressing to the recipient site (e.g., periwound area) in order to hold the transfer dressing in place and/or facilitate handling of the transfer dressing.

In one aspect, a transfer dressing for transplanting a skin graft is disclosed. The transfer dressing can comprise a substrate having at least one surface configured to receive an excised skin graft and engage said skin graft for removal from a donor site, and at least one tacky appendage, coupled to a portion of the substrate, and configured to secure the substrate to at least a portion of skin adjacent to a recipient site.

In another aspect, a method for transplanting a skin graft is disclosed. The disclosed method comprises placing a surface of a transfer substrate of a transfer dressing carrying one or more skin grafts on a recipient skin site of a patient. The transfer dressing can comprise at least one tacky appendage foldably coupled to said substrate. The method further includes attaching the tacky appendage to a portion of the patient's skin adjacent to the recipient site so as to secure the transfer dressing to the patient's skin.

In other examples, any of the aspects above, or any system, method, apparatus described herein can include one or more of the following features.

The tacky appendage can be coupled to a peripheral portion of the substrate. Additionally or alternatively, the tacky appendage can be foldably coupled to the substrate. Further, the appendage can be configured to be foldable to rest on a surface of the substrate opposed to the at least one surface configured to receive the excised skin graft. The tacky appendage can also be configured to be foldable onto itself.

The substrate can be configured to fit in a chamber of a skin graft harvesting device. Further, in some embodiments, the at least one surface of the substrate for receiving the skin graft can comprise a plurality of fenestrations. The fenestrations can have a minimum width of about 3 micrometers. In some embodiments, at least one fenestration can have a size approximately 20% smaller than a length of the at least one surface. Alternatively or additionally, at least one fenestration can have a size approximately equal to a length of the at least one surface. The fenestrations can have a plurality of different shapes. For example, the fenestrations can be round-shaped, square-shaped, oblong-shaped, rectangular-shaped, star-shaped, or a combination thereof. Further, the at least one surface of the substrate can comprise at least one of a meshed configuration, a porous configuration, a weaved configuration, a film having one or more slits, or a combination thereof. Generally, the substrate can comprise at least one of a thermoplastic polymer, acrylic, polyester, thermoplastic silicon, polyester urethane, polyether urethane, or a combination thereof. Further, the substrate surface can comprise an adhesive. The adhesive can include at least one of an acrylate, a polyurethane, silicon, one or more silicon-based materials, natural rubber, a synthetic rubber, a polyolefin, a styrene-based block copolymer, a hydrogel, a hydrocolloid, or a combination thereof. The substrate can have a surface density ranging from about 10 grams/meter² to about 100 grams/meter².

In some embodiments, the at least one tacky appendage is configured such that it can be wrapped around a region of a patient's body adjacent to the recipient site. By way of example, the appendage can be configured to be square-shaped, rounded, triangle-shaped, or a combination thereof. In some embodiments, the at least one appendage can have a width ranging from about 1% of a width of the substrate to about 100% of the width of the substrate.

Further, the appendage can comprise an adhesively-coated surface for securing the appendage to the at least a portion of a patient's skin adjacent to the recipient site. A releasable liner can be coupled to the adhesively-coated surface. The releasable liner can be configured to be manually removable from the adhesively-coated surface.

In some embodiments, the tacky appendage can comprise two appendages that are configured to secure the transfer dressing to the patient's skin via attaching one appendage to another.

In some implementations, the transfer dressing can further comprise a carrier film coupled to the substrate to provide mechanical support to the substrate. The carrier film can comprise at least one of polypropylene, polyethylene, polyether urethane, thermoplastic silicone, or a combination thereof.

Other aspects and advantages of the invention can become apparent from the following drawings and description, all of which illustrate the principles of the invention, by way of example only.

### BRIEF DESCRIPTION OF THE DRAWINGS

Features and advantages of the invention described herein, together with further advantages, may be better understood by referring to the following description taken in conjunction with the accompanying drawings. The drawings are not necessarily to scale, emphasis instead is generally placed upon illustrating the principles of the invention.

FIG. 1A is a schematic illustration of a transfer dressing according to some embodiments disclosed herein.

FIG. 1B is an expanded view of a portion of FIG. 1A that schematically illustrates an example embodiment of a lower surface of the transfer dressing

FIGS. 2A-2D schematically illustrate examples of structures that can be used with the embodiments disclosed herein for capturing skin grafts.

FIGS. 3A-3B schematically illustrate an example transfer dressing according to some embodiments described herein.

FIGS. 4A-4B schematically illustrate another example transfer dressing according to some embodiments described herein.

FIGS. 5A-5B schematically illustrate yet another example transfer dressing according to some embodiments described herein.

FIGS. 6A-6C illustrate an example of a prototype transfer dressing according to some embodiments disclosed herein.

FIGS. 6D-6F illustrate another examples of a prototype transfer dressing according to some embodiments disclosed herein.

FIG. 7A-7L illustrate example procedures that can be followed in order to harvest and transfer epidermal skin grafts from a simulated donor site to a simulated recipient site using a transfer dressing configured according to some embodiments disclosed herein.

### DETAILED DESCRIPTION

The present disclosure generally relates to an epidermal skin graft transfer dressing that is configured to prevent undesired movement of the transfer substrate (e.g., movements into tight folds, bunching up, etc.) by using built-in adhesive extensions or appendages that are configured to secure the transfer dressing to the periwound area by holding the dressing securely in place.

A transfer dressing according to some embodiments described herein can be used in conjunction with a skin micrograft harvester, such as the CelluTome® harvester, to capture and retain a plurality of skin micrografts (or microdomes). The transfer dressing can utilize a flexible transfer substrate (e.g., a flexible mesh) that comprises and/or is coated with a suitable material. For example, the flexible substrate can comprise and/or be coated with a material, such as silicone (e.g., a silicone gel) or polyurethane (PU). Further, the substrate can be adhesive and/or have a soft tack, which when pressed onto microdomes, allows for the slight immersion of the top of the microdome into the substrate and enables the removal of the microdomes from the harvester.

It should be noted that the terms “soft-tack” and/or “tacky,” as used herein, refer to the ability of a surface to bind to other surfaces or objects in a more releasable and gentler manner than conventional adhesives. Further, the terms “micrograft” and “microdome” are used interchangeably herein and are intended to encompass skin grafts that have a width or length less than about a millimeter, more preferably, less than about 100 microns. A micrograft or microdome is an excised skin segment having at least one dimension parallel to the skin surface that is at least one of: less than a millimeter, less than 100 micrometers, and/or less than 10 micrometers. The minimum width or length can be at least one of: less than 500 micrometers, less than 100 micrometers, less than 50 micrometers, less than 10 micrometers, and/or less than 1 micrometer. A micrograft or microdome can also assume any suitable shape. For example, a micrograft or microdome can be generally circular, oval or oblong in a plane parallel to the skin surface and have a diameter or major axis that ranges from about 1 millimeter to 0.01 micrometers, from about 100 micrometers to about 0.1 micrometers, from about 50 to 1 micrometers. Micrografts and microdomes can also have a depth dimension that extends at least through the epidermis and, in some applications, encompasses at least one layer of basal cells. The depth can range from about 500 micrometers to about 0.1 micrometers and/or from about 100 micrometers to about 1 micrometer.

Furthermore, the term “harvesting,” as used herein, is intended to encompass the removal of one or more skin grafts from a skin graft generating device (e.g., a suction blister micrograft generator) and any intermediate steps (e.g., culturing, expanding, stretching, treating or otherwise preparing a skin graft for transfer to a recipient site).

Still further, the term “dressing” is used herein to refer to a transfer dressing that includes a substrate generally used not only to capture excised skin grafts but also to retain them for transplantation. During the transplantation process, the substrate (or dressing), along its captured grafts, can be applied directly to a recipient site. Both substrates and dressings can also encompass other elements in addition to a soft-tack, porous surface, e.g., fluid absorbent layers or cap layers.

Yet further, the terms “porous,” “perforated,” and/or “fenestrated,” as used herein, are used to described any suitable material having pores, mesh, weave, film with slits. Further, the terms “pores,” “mesh,” “weave,” “film with slits” are intended to encompass not only apertures or holes but also permeable and open cell structures. In certain embodiments, a permeable or porous composition can be formed from woven or non-woven (e.g., matted) fibers. The fibrous base layer can include microfibers and/or nanofibers.

Furthermore, the term “about,” as used herein, refers to variations in a numerical quantity that can occur, for example, through measuring or handling procedures in the real world, through inadvertent error in these procedures, through differences in the manufacture, source, or purity of compositions or reagents; and the like. Typically, the term “about” as used herein means greater or lesser than the value or range of values stated by 1/10 of the stated values, e.g., ±10%. For instance, a concentration value of about 30% can mean a concentration between 27% and 33%. The term “about” also refers to variations that would be recognized by one skilled in the art as being equivalent so long as such variations do not encompass known values practiced by the prior art. Each value or range of values preceded by the term “about” is also intended to encompass the embodiment of the stated absolute value or range of values. Whether or not modified by the term “about,” quantitative values recited in the claims include equivalents to the recited values, e.g., variations in the numerical quantity of such values that can occur, but would be recognized to be equivalents by a person skilled in the art.

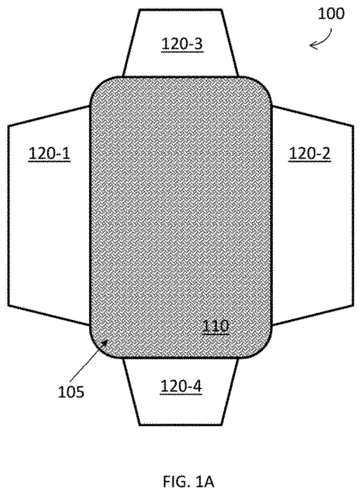

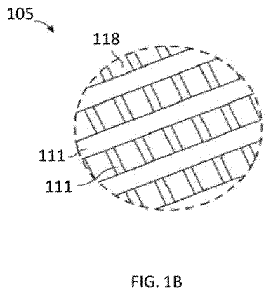

FIG. 1A is a schematic illustration of a transfer dressing 100 according to some embodiments disclosed herein. The example transfer dressing 100 is shown in FIG. 1 on its back or distal side, which is the side of the transfer dressing/substrate 100 that comes in contact with the patient's skin. As shown, the transfer dressing 100 can include a transfer substrate 110, which has a tacky lower surface 105, and one or more peripheral appendages 120-1, . . . , 120-4 attached to a periphery of at least one side of the substrate 110.

FIG. 1B is an expanded view of a portion of FIG. 1A that schematically illustrates an embodiment of the lower surface 105. As shown, the lower surface 105 of the transfer substrate 110 can include a plurality of sites 118 for capturing skin grafts. For example, the lower surface 105 can include a porous, perforated, and/or fenestrated surface structures 111 disposed between the sites for capturing skin grafts. Generally, any suitable perforated or fenestrated material that contains the appropriate structure 111 for receiving wound exudates can be used. For example, the lower surface 105 can comprise any suitable material having a porous, mesh-shaped, or weave-shaped structure, any suitable film having one or more slits to manage wound exudates, or a combination thereof.

The fenestrations and/or perforations structures 111 included in the lower surface 105 can have any suitable size and/or shape. For example, the size of the fenestrations and/or perforations 111 can range from 3 μm to an upper fenestration size limit that is equal to the size of the transferable epidermal skin graft. In some embodiment, the fenestrations and/or perforations 111 can be about the same size as the smallest available pore size that can allow for fluid transfer. Further, in some embodiments, the size of the fenestrations and/or perforations 111 can be arranged such that the fenestrations and/or perforations 111 are about 20% smaller than the size of the epidermal skin graft being transferred.

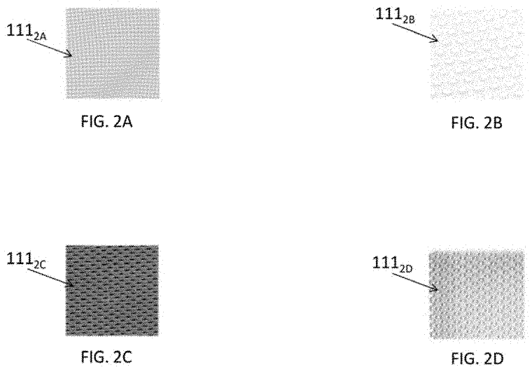

Further, as noted, the fenestrations and/or perforations 111 can have any suitable shape. For example, the fenestrations and/or perforations can be circular, round, square, oblong, rectangular, star shaped, amorphous, etc. in one or more dimensions. FIGS. 2A-2D illustrate examples of fenestrations and/or perforations 111 that can be used with the embodiments disclosed herein. As shown, the fenestrations and/or perforations 111 can be box-shaped 111_{2A} (FIG. 2A), oblong 111_{2B} (FIG. 2B), rectangular-shaped 111_{2C} (FIG. 2C), and/or round 111_{2D} (FIG. 2D). Generally, the shape or size of the pores 111 is configured to ensure that the porosity of the substrate 110 is sufficient to permit fluid migration from a skin segment (e.g., a segment of skin at the recipient site) through the surface 105 of the substrate 110.

Furthermore, the transfer substrate 110 can be a soft-tack or tacky substrate. It should be noted that the terms “soft-tack” and “tacky,” as used herein, refer to the ability of a surface to bind to other surfaces or objects in a more releasable and gentler manner than conventional adhesives. The degree of tackiness can be measured by any suitable/available means, for example the loop tack test.

For example, the transfer substrate 110 can be formed of a soft material suitable for both capturing micrografts and providing a fluid seal with the skin graft transplantation site. Generally, the transfer substrate 110 can comprise any suitable material. For example, the substrate 110 can comprise at least one of: a silicone gel, a soft silicone, hydrocolloid, hydrogel, polyurethane gel, polyolefin gel, hydrogenated styrenic copolymer gels, a foamed gel, a soft closed cell foam such as polyurethanes and polyolefins, polyurethane, polyolefin, or hydrogenated styrenic copolymers coated with an adhesive (described below). The transfer substrate 110 can have any suitable thickness or stiffness. In some embodiments, the transfer substrate 110 can have a surface density ranging from about 10 grams/meter² to about 100 grams/meter².

Additionally, in some embodiments, the transfer substrate 110 can be a hydrophobic-coated material. For example, the transfer substrate 110 can be formed by coating a fenestrated and/or perforated material (e.g., woven, nonwoven, molded, or extruded mesh) with a hydrophobic material. The hydrophobic material for the coating can be any suitable material, such as soft silicone. Various factors can be considered in controlling the ability of the substrate to capture skin grafts. Such factors include, for example, the diameter and number of the fenestrations and/or perforations 111 in the substrate 110, the thickness of the substrate 110, and the tackiness of the substrate 110.

Referring back to FIG. 1A, the fenestrated and/or perforated (hereinafter collectively “fenestrated”) surface 105 of the transfer substrate 110 can transfer autologous epidermal skin grafts to a graft recipient site (not shown) via any suitable means. For example, as described in further details below, the fenestrated surface 105 can be an adhesive film having an adhesive that facilitates adhesion of the fenestrated surface 105 to the recipient site. Once placed at the recipient site, the transfer dressing 100 can be left at the recipient site for a period of time to allow for starting of the re-epithelialization process.

The adhesive film can comprise any suitable adhesive. For example, the adhesive film can comprise one or more thermoplastic polymers, such as acrylics, polyester, thermoplastic silicone, polyester urethanes, polyether urethane, or their combinations. Additionally or alternatively, the adhesive can comprise one or more materials including, but not limited to, acrylates, polyurethanes, silicone, or silicone based materials, natural or synthetic rubber, polyolefins, styrene based block copolymers, hydrogels and hydrocolloids.

The thickness of the adhesive film can depend on various factors such as the type of adhesive used and the required end applications (e.g., the ability of the substrate to capture skin grafts). For example, the adhesive film can have a thickness ranging from 10 to 200 grams per square meter. Depending on film thickness and mechanical properties, the transfer dressing film can be supported using a carrier film (not shown). The carrier film can comprise any suitable material. For example, the carrier film can comprise at least one of polypropylene, polyethylene, polyetherurethane, or thermoplastic silicone.

Generally, the transfer dressing 100 and/or the adhesive film are arranged such that the transfer dressing 100, while having sufficient adhesive strength for transferring the epidermal skin grafts, is generally non-adherent to the wound bed. Specifically, the transfer dressing 100 can be arranged such that it has the adhesive strength sufficient for transferring at least 50% of the total epidermal skin grafts within an array for transfer, while being generally non-adherent to the wound bed, once grafted, to avoid traumatic release from the wound bed upon dressing removal (e.g., at 3-14 days post grafting). In other words, the transfer substrate 110 can have sufficient adhesive strength to allow the capture of the skin grafts but is not so adhesive. In addition to having the appropriate adhesive strength to transfer the epidermal grafts, the characteristics of the base materials used to create the transfer dressing can be formulated and processed with the ability to foster, at the minimum, cellular migration and proliferation.

Referring back to FIG. 1A, as noted above, the transfer dressing 100 can include one or more appendages 120-i, i=1, 2, 3, . . . that are configured to facilitate attachment of the transfer dressing 100 to the recipient site. Specifically, the one or more appendages 120-i can be configured such that they can secure the transfer dressing 100 to at least a portion of the patient's body at the recipient site.

The one or more appendages 120-i can be coupled to at least a portion of the transfer dressing 100. For example, the one or more appendages 120-i can be attached to at least a portion of the perimeter of the transfer dressing 100. Additionally or alternatively, in some embodiments, at least one appendage 120-i can be connected or coupled to a portion of the transfer dressing. For example, in some implementations, at least one appendage 120-i can be coupled to a portion of the front side of the transfer dressing (e.g., the side of transfer dressing positioned opposite the fenestrated side). Further, the appendages 120-I can be configured as solid border extension that extend from the outer boundary of the transfer substrate 110 and/or be individual pieces that extend beyond the perforated center of the transfer substrate 110. In some embodiments, the appendages can be connected to one another such that they form a contiguous surface.

Generally, any number of appendages 120-i can be used. Further, the appendages 120-i can assume any suitable shape or size. The number of appendages 120-i used and/or the size and/or shape of the appendages 120-i can depend on various factors, such as the size of the recipient site, the location of the recipient site, etc. In the example shown in FIG. 1A, the transfer dressing 100 is shown as having four trapezoid shaped appendages 120-1, 120-2, 120-3, 120-4. However, any suitable number of appendages 120-i can be used.

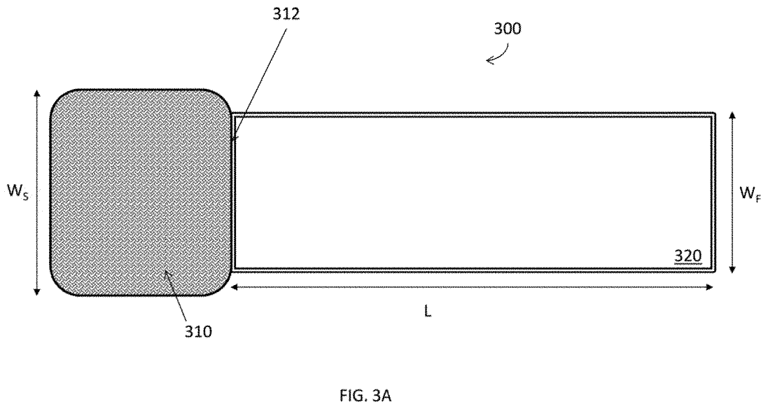

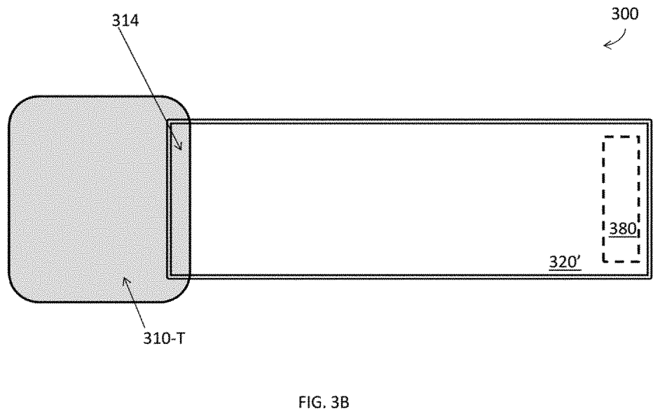

FIG. 3A is a schematic illustration of an example transfer dressing 300 according to some embodiments described herein. As shown in FIG. 3A, in one embodiment, the transfer dressing 300 can comprise a fenestrated transfer substrate 310 that has been coupled to an appendage 320. In the example shown in FIG. 3A, the appendage 320 is shown as having been coupled to the transfer substrate 310, on one side of the transfer substrate 312, along the external boundary (i.e., perimeter) of the transfer substrate 310. However, as noted above, the appendage 320 can connect/be coupled to the transfer substrate 310 at any suitable position/location. For example, as shown in FIG. 3B, an appendage 320′ can be attached to a portion 314 of the transfer substrate, for example to a portion of the side 310-T of the transfer substrate disposed opposite the fenestrated side.

In the example shown in FIGS. 3A-3B, the fenestrated transfer substrate 310 is coupled to a single appendage 320. Generally, the appendage 320 can be of any suitable shape or length. For example, the appendage 320 can be an elongated appendage 320 configured to be capable of being wrapped around a body part of the patient (e.g., a leg or arm of the patient). Once wrapped around the patient's body part, the elongated appendage 320 can be secured in place using any suitable technique. For example, the elongated appendage 320 can be clamped, taped, or secured using any other suitable technique. In the example shown in FIG. 3B, the elongated appendage 320′ includes an adhesive patch 380, which can be used to secure the elongated appendage 320 in place to prevent the appendage 320′ from unraveling after being wrapped around the body part. Alternatively or additionally, in some embodiments, the appendage 320 can have one or more adhesive borders that facilitate adhesion of the appendage against the patient's body and/or prevent unraveling of the appendage, for example by securing the appendage onto itself.

In other embodiments, the transfer dressing can include any number of appendages. For example, as shown in FIG. 1A, the transfer dressing 100 can include four appendages 120-1, . . . , 120-4 positioned on all four sides of a square or rectangular transfer substrate. In some embodiments, the transfer dressing can include two or three appendages and/or more than four appendages. Further, although shown as having a single appendage 120-1, . . . , 120-4 on each side of the transfer substrate 110, the transfer dressing 100 can include two or more appendages coupled to and/or extending from each side thereof 110. Furthermore, the transfer substrate 110 can be a circular or elliptical shape and have one or more appendages coupled to and/or extending from its perimeter. The transfer dressing 100 can also assume any suitable shape known in the art.

Referring to FIG. 3A, generally, the appendages have any suitable width W_{F}, length L, and/or thickness. For example, each appendage can have a width W_{F}, ranging from about 1% of the width W_{S} of the transfer substrate 310 to about 100% of the width W_{S} of the transfer substrate 310. In embodiments that include the transfer substrate having shapes such as circular or elliptical shapes, each appendage can have a width ranging from about 1% of the diameter of the transfer substrate to about 100% of the diameter of the transfer substrate. In some embodiments, the appendages can be arranged such that they secure the transfer dressing against the recipient site through attachment of one or more appendages to one or more other appendages.

Further, the appendages can be of any suitable shape. For example, the appendages can be rectangular, square-shaped, elliptical, circular, or any other suitable shape. In some embodiments, the appendages can be angled to allow for folding of the appendages back onto the transfer substrate.

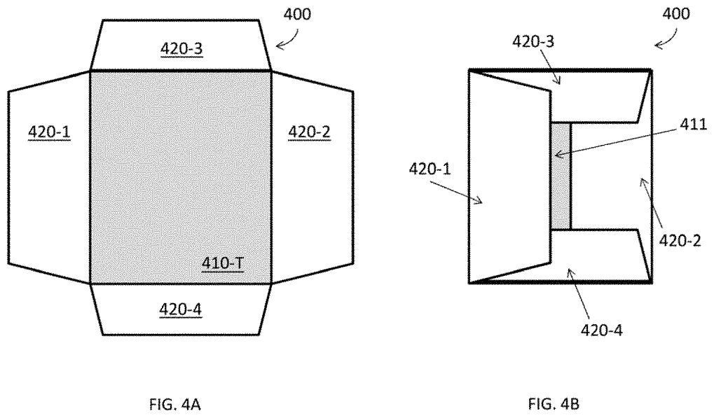

FIGS. 4A-4B schematically illustrate an embodiment of the transfer dressing 400 having appendages that can be folded onto the transfer substrate. In FIG. 4A, the transfer substrate is shown on its top side 410-T (i.e., proximal side, side positioned opposite its fenestrate side/side positioned opposite the side that comes directly in contact with the recipient and/or donor site). As shown, in this example, the transfer dressing 400 includes four generally trapezoid-shaped appendages 420-1, . . . , 420-4 disposed along the boundary of the transfer substrate 410-T. The appendages 420-1, . . . , 420-4 can be angled and arranged such that they are foldable about the boundary of the transfer substrate 410-T and can be folded onto the transfer substrate 410-T. FIG. 4A illustrates the appendages 420-1, . . . , 420-4 in the open/extended configuration, while FIG. 4B illustrates the appendages in the folded configuration. As noted above, the appendages 420-1, . . . , 420-4 can have various sizes and shapes. In some embodiments, the appendages 420-1, . . . , 420-4 can be arranged such that each appendage can be folded more one or more times onto itself. Further, the appendages can each be configured such that after being folded, each appendage extends beyond the center of the transfer substrate 410-T.

By being foldable onto and along the boundary of the transfer substrate 410-T, the appendages 420-1, . . . , 420-4, facilitate usage of the transfer substrate 410-T and the transfer dressing 400 with harvester devices, such as the CelluTome® skin harvester (available from Acelity, Inc. of San Antonio, Tex.).

Specifically, the transfer dressing 400 can be configured such that the appendages 420-1, . . . , 420-4 can be folded onto the transfer substrate 410-T and/or onto themselves (i.e., each appendage can be folded onto itself) to allow for fitting of the transfer dressing 410-T into a skin harvester (not shown). Once in the folded configuration (FIG. 4B), the transfer dressing 400 can be placed in the harvester such that the fenestrated side of the transfer dressing 100 comes in contact with the donor site on the patient's skin (e.g., the blister's formed on the skin at the donor site). Once harvesting is complete, the transfer dressing 400 can be removed from the harvester and transferred to a recipient site on the patient's body. The transfer dressing 400 can be removed and/or transferred using the appendages. For example, a clinician or practitioner can use the appendages to lift and transfer the transfer dressing 400 to the recipient site on the patient's body. For example, to transfer the transfer dressing 400, the practitioner can lift and unfold the appendages 420-1, . . . , 420-4 and use one of more of the appendages to hold and transfer the dressing to the recipient site.

Once transferred, the fenestrated transfer dressing 400 can protect the epidermal skin grafts by providing a physical protective barrier that secures the integrity of migrating and proliferating keratinocytes by preventing loss of contact between the grafts and the wound bed via dressing folding and/or bunching using the one or more appendages 420-1, . . . , 420-4. Further, as noted above, in some embodiments, the appendages 420-1, . . . , 420-4 can be adhesive extensions that are configured to adhere tightly to periwound skin, thereby preventing the transfer dressing 400 from folding and/or bunching.

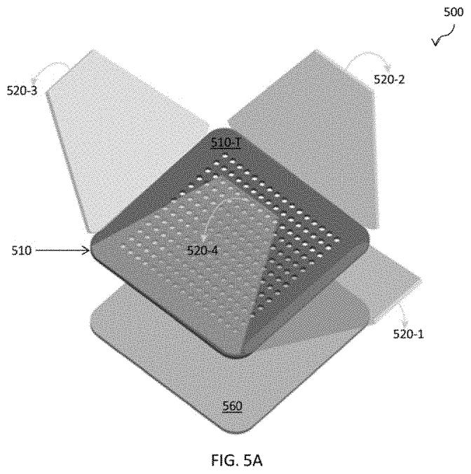

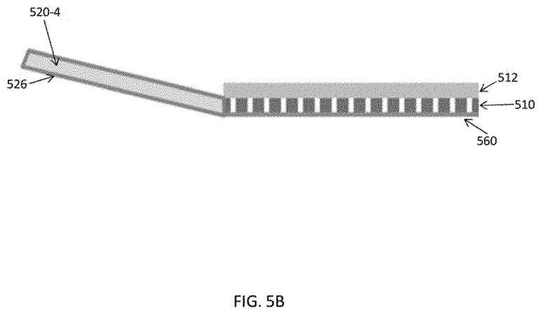

FIGS. 5A-5B schematically illustrate yet another example transfer dressing 500 according to some embodiments described herein. As shown in FIG. 5A, the transfer dressing 500 includes a fenestrated transfer substrate 510. The transfer dressing 510 can include a top side 510-T, onto which the foldable appendages 520-1, . . . , 520-4 can be folded. As noted above, the appendages 520-1, . . . , 520-4 can comprise and/or include an adhesive that facilitates attachment of the appendages 520-1, . . . , 520-4 to the areas surrounding the recipient site (e.g., periwound area). As shown in FIG. 5, and described above, the appendages 520-1, . . . , 520-4 can be foldable such that they can be folded onto the top surface of the transfer substrate 510-T (e.g., for placement in a skin harvester) and can be folded away from the top surface of the transfer substrate 510-T (e.g., for securing the transfer dressing 510 against the recipient site and/or to prevent the transfer dressing from bunching and/or folding).

As noted above, the transfer substrate 510 can also comprise a tacky or adhesive material configured to facilitate harvesting of epidermal skin grafts and attachment of the transfer dressing 510 to the recipient site. The tacky or adhesive material can be positioned on the side of the transfer substrate that comes in contact with the recipient site (e.g., the side of the substrate opposite the top side 510-T). Generally, any tacky and/or adhesive material known in the art can be used (e.g., tacky silicon). The transfer dressing 510 can comprise and/or be coupled to a release liner 560. The release liner 560 can be a removable release liner 560. Specifically, the release liner 560 can be configured such that it can be removed to expose the tacky or adhesive side of the transfer substrate 510. In use, the release liner 560 can be removed to expose the side of the substrate that comes in contact with the patient's skin.

The transfer substrate 510 and/or the appendages can include various other structures. For example, as shown in FIG. 5B, the transfer substrate 510 can include a removable support layer 512. The support layer 512 and the releasable liner 560 can be applied to the upper and lower surfaces of the transfer substrate 510, respectively.

In use, the lower release liner 560 can be arranged such that it can be removable to expose the less tacky lower surface of the substrate 510. Specifically, the lower release liner 560 can be arranged such that it is relatively easily and selectively removable due to the lower adherence of this sheet to the material compared to the upper support layer 512. The lower and/or upper release layer can further comprise indicia to identify the release sheet to be removed first. Upon removal of the lower release liner 560, the lower surface of the transfer substrate 110 can be applied to a skin graft harvester bed, followed by removal of the upper support layer 512 at the time of removal from the harvester or transplantation (or at the time of application of optional secondary dressing elements such as an absorbent layer, if desired).

Further, in certain embodiments, at least one of the appendages can include a removable release liner 526. Although, only one appendage 520-4 is shown in FIG. 5B, it should be understood that any/all of the appendages can include a releasable liner. The releasable liner 526 can be positioned on either and/or both sides of each appendage 520-4. For example, the releasable liner 526 can be positioned on a side of the appendage 520-4 that comprises an adhesive. Specifically, the releasable liner 526 can be arranged such that it covers the adhesive side of its corresponding appendage 520-4 and/or on an adhesive patch disposed on a side of the appendage and can be removed to expose the adhesive side/portion of the appendage 520-4.

Transfer dressings according to the embodiments disclosed herein can provide for optimized transfer of skin grafts (e.g., skin autografts comprised of human epidermal cells) to facilitate the regrowth of epidermal cells on areas denuded of native skin structures (e.g., for the treatment of vitiligo patients, for treatment of ulcers, burn patients, treatment of mucosal lesions, and skin closure after surgical removal of skin cancers).

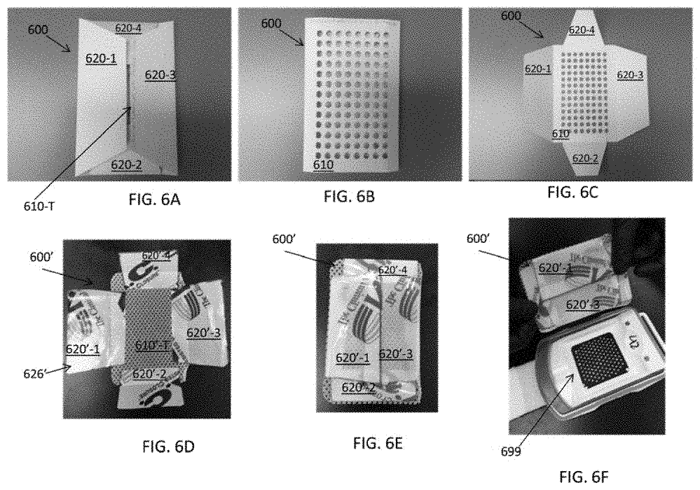

FIGS. 6A-6C illustrate an example of a prototype transfer dressing 600 according to some embodiments disclosed herein. FIGS. 6D-6F illustrate another example of a prototype transfer dressing according to some embodiments disclosed herein. Specifically, FIG. 6A illustrates a prototype transfer dressing 600 having foldable appendages 620-1, . . . , 620-4 that have been folded onto the top surface 610-T of the transfer substrate of the transfer dressing 600. FIG. 6B illustrates the prototype transfer dressing on its back side, with the fenestrated portion of the transfer substrate 610 exposed and ready for placement into a skin harvester, such as a CelluTome® device. FIG. 6C illustrates the prototype transfer dressing 600 after the appendages 620-1, . . . , 620-4 have been unfolded and are, hence, in their extended form.

FIGS. 6D-6F illustrate another examples of a prototype transfer dressing 600′ according to some embodiments disclosed herein. As shown in FIG. 6D, the transfer dressing 600′ can include a transfer substrate from which a plurality of appendages 620′-1, . . . , 620′-4 extend (FIG. 6D illustrates the top view 610′-T of the transfer substrate). The appendages 620′-1, . . . , 620′-4 can include an adhesive material that facilitates adhesion of the appendages 620′-1, . . . , 620′-4 to the periwound area and/or the area surrounding the recipient area on the patient's body. The appendages 620′-1, . . . , 620′-4 can include a removable liner 626′ that can be removed to expose the adhesive portions prior to the application of the appendages to the periwound area and/or the area surrounding the recipient area on the patient's body. As shown in FIG. 6E, the appendages 620′-1, . . . , 620′-4 can be configured such that they can be folded onto the transfer substrate (the top surface of the transfer substrate 610′-T). As shown in FIG. 6F, once folded onto the transfer substrate, the transfer dressing 600′ can be placed in a skin harvester, such as the CelluTome® device 699. The transfer dressing 600′ can be placed in the CelluTome® device 699 such the fenestrated surface of the transfer substrate can come into contact with the donor site.

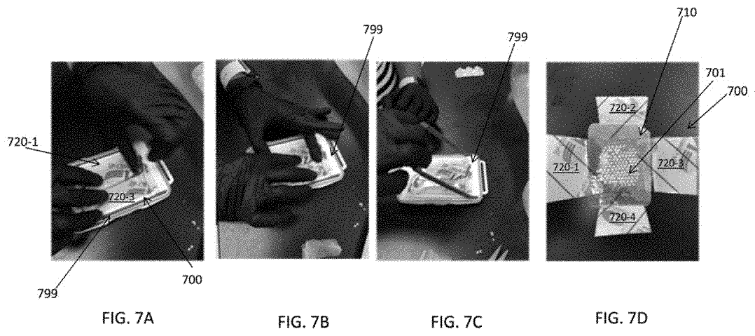

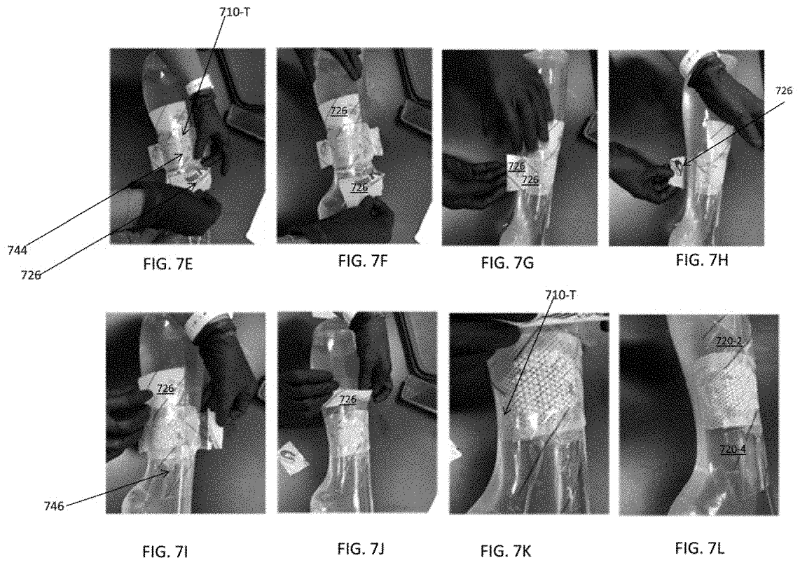

FIGS. 7A-7L illustrate example procedures that can be followed in order to harvest and transfer epidermal skin grafts from a simulated donor site to a simulated recipient site using a transfer dressing 700 configured according to some embodiments disclosed herein. As shown in FIG. 7A, the transfer dressing 700 can be placed in a skin harvester (e.g., the CelluTome® device 799) with the appendages 720-1, . . . , 720-4 of the transfer dressing folded onto the top surface of the transfer substrate 710-T. The transfer dressing 700 can be arranged such that after the appendages 720-1, . . . , 720-4 are folded onto its top surface it can fit within the CelluTome® harvester 799. For example, the transfer dressing 700 can be a fenestrated synthetic transfer dressing to fit the CelluTome® harvester within +/−10% of the size of any described CelluTome® harvester. Further, as noted above, the side of the transfer dressing 700 that comes in contact with the donor site can be an adhesive or tacky surface that facilitates the harvesting of the epidermal grafts. The CelluTome® harvester can then be used to separate epidermis from dermis using negative pressure and warmth with the epidermal harvesting system, CelluTome®. Raised epidermis is then placed in direct contact with the fenestrated transfer dressing portion. Additional details on harvesters useful in connection with the present invention can be found in U.S. patent application Ser. No. 13/839,518 filed Mar. 15, 2013; U.S. patent application Ser. No. 13/346,329 filed Jan. 9, 2012; U.S. patent application Ser. No. 13/436,318 also filed Jan. 9, 2012; U.S. patent application Ser. No. 13/014,737 filed Jan. 27, 2011; U.S. patent application Ser. No. 12/851,656 filed Aug. 6, 2010; U.S. patent application Ser. No. 12/851,621 filed Aug. 6, 2010; U.S. patent application Ser. No. 12/851,703 filed Aug. 6, 2010; and U.S. patent application Ser. No. 12/851,682 filed Aug. 6, 2010. The contents of each of the above-referenced related applications are herein incorporated by reference in their entireties.

As shown in FIG. 7C, once harvesting is complete, the CelluTome® harvester is actuated to cut the harvested epidermal skin grafts 701 (FIG. 7D). The epidermis can be cut away from the dermis with the fenestrated synthetic transfer dressing adhered in place to maintain epidermal cell orientation.

Extension appendages 720-1, . . . , 720-4 can be extended to reveal the epidermal grafts 701 transfer rate using the fenestrated center portion (transfer substrate 710) of the transfer dressing 700.

As shown in FIGS. 7E-7L, the perforated transfer dressing 700 can be placed on the wound bed 744 (reference number 744 is used to generally refer to the simulated wound bed under the fenestrated portion 710 that includes the simulated epidermal grafts 701). Release liners 726 on the adhesive appendages 720-1, . . . , 720-4 can then be removed and the adhesive appendages 720-1, . . . , 720-4 can be placed over periwound skin 746 (reference number 746 is used to refer to the portion of the simulated skin positioned under the appendages 720-1, . . . , 720-4 and adjacent to the simulated wound bed 744). Once in place, the fenestrated synthetic transfer dressing 700 can protect and sustain epidermal cell integrity at the recipient site. The appendages 720-1, . . . , 720-4 maintain the transfer substrate 710 in place and prevent the transfer substrate 710 from bunching and/or folding.

Although this specification discloses advantages in the context of certain illustrative, non-limiting embodiments, various changes, substitutions, permutations, and alterations may be made without departing from the scope of the specification as defined by the appended claims. Further, any feature described in connection with any one embodiment may also be applicable to any other embodiment.

1. A transfer dressing for transplanting a skin graft, the transfer dressing comprising:
a substrate having at least one surface configured to receive an excised skin graft and engage said skin graft for removal from a donor site; and
at least one tacky appendage, coupled to a portion of the substrate, and configured to secure the substrate to at least a portion of skin adjacent to a recipient site.

2. The transfer dressing of claim 1, wherein the at least one tacky appendage is coupled to a peripheral portion of the substrate.

3. The transfer dressing of claim 1, wherein said at least one tacky appendage is foldably coupled to said substrate.

4. The transfer dressing of claim 3, wherein said at least one appendage is configured to be foldable to rest on a surface of the substrate opposed to said at least one surface configured to receive the excised skin graft.

5. The transfer dressing of claim 1, wherein the substrate is configured to fit in a chamber of a skin graft harvesting device.

6. The transfer dressing of claim 1, wherein the at least one tacky appendage is configured to be foldable onto itself.

7. The transfer dressing of claim 1, wherein said at least one appendage has a width ranging from about 1% of a width of the substrate to about 100% of the width of the substrate.

8. The transfer dressing of claim 1, wherein said at least one tacky appendage is configured such that it can be wrapped around a region of a patient's body adjacent to the recipient site.

9. The transfer dressing of claim 1, wherein said appendage is configured to be square-shaped, rounded, triangle-shaped, or a combination thereof.

10. The transfer dressing of claim 1, wherein said at least one appendage comprises an adhesively-coated surface for securing said appendage to the at least a portion of a patient's skin adjacent to the recipient site.

11. The transfer dressing of claim 10, further comprising a releasable liner coupled to the adhesively-coated surface, the releasable liner being configured to be manually removable from the adhesively-coated surface.

12. The transfer dressing of claim 1, wherein said at least one tacky appendage comprises two appendages configured to secure the transfer dressing to the patient's skin via attaching one appendage to another.

13. The transfer dressing of claim 1, wherein said at least one surface of the substrate for receiving the skin graft comprises a plurality of fenestrations.

14. The transfer dressing of claim 13, wherein said fenestrations have a minimum width of about 3 micrometers.

15. The transfer dressing of claim 13, wherein at least one fenestration has a size approximately 20% smaller than a length of the at least one surface.

16. The transfer dressing of claim 13, wherein at least one fenestration has a size approximately equal to a length of the at least one surface.

17. The transfer dressing of claim 13, wherein at least one fenestration is round-shaped, square-shaped, oblong-shaped, rectangular-shaped, star-shaped, or a combination thereof.

18. The transfer dressing of claim 1, wherein the at least one surface comprises at least one of a meshed configuration, a porous configuration, a weaved configuration, a film having one or more slits, or a combination thereof.

19. The transfer dressing of claim 1, wherein the substrate comprises at least one of a thermoplastic polymer, acrylic, polyester, thermoplastic silicon, polyester urethane, polyether urethane, or a combination thereof.

20. The transfer dressing of claim 1, wherein said substrate surface comprises an adhesive.

21. The transfer dressing of claim 20, wherein the adhesive includes at least one of an acrylate, a polyurethane, silicon, one or more silicon-based materials, natural rubber, a synthetic rubber, a polyolefin, a styrene-based block copolymer, a hydrogel, a hydrocolloid, or a combination thereof.

22. The transfer dressing of claim 1, wherein the substrate has a surface density ranging from about 10 grams/meter² to about 100 grams/meter².

23. The transfer dressing of claim 1, further comprising a carrier film coupled to the substrate to provide mechanical support to the substrate.

24. The transfer dressing of claim 23, wherein the carrier film comprises at least one of polypropylene, polyethylene, polyether urethane, thermoplastic silicone, or a combination thereof.

25. A method for transplanting a skin graft, the method comprising:
placing a surface of a substrate of a transfer dressing carrying one or more skin grafts on a recipient skin site of a patient, said transfer dressing comprising at least one tacky appendage foldably coupled to said substrate, and
attaching said tacky appendage to a portion of the patient's skin adjacent to said recipient site so as to secure the transfer dressing to the patient's skin.

26-45. (canceled)
